(19) United States
(12) Patent Application Publication
(10) Pub. No.: US 2013/0213154 A1
(43) Pub. Date: Aug. 22, 2013

(54) Method of Determining Cleanliness

(71) Applicants: Timothy M. Crowder, Research Triangle Park, NC (US); Jason E. Dickens, Research Triangle Park, NC (US); Kevin J. Ely, Research Triangle Park, NC (US); Dwight Sherod Walker, Research Triangle Park, NC (US)

(72) Inventors: Timothy M. Crowder, Research Triangle Park, NC (US); Jason E. Dickens, Research Triangle Park, NC (US); Kevin J. Ely, Research Triangle Park, NC (US); Dwight Sherod Walker, Research Triangle Park, NC (US)

(73) Assignee: GLAXO GROUP LIMITED, GREENFORD MIDDLESEX (GB)

(21) Appl. No.: 13/880,402

(22) PCT Filed: Nov. 9, 2011
(86) PCT No.: PCT/US11/59849

§ 371 Date: Apr. 19, 2013

Related U.S. Application Data
(60) Provisional application No. 61/412,151, filed on Nov. 10, 2010.

(51) Int. Cl. G01N 21/64; G01N 21/25; G01N 21/65; G01N 33/00

(52) U.S. Cl. CPC G01N 21/64; G01N 33/00; G01N 21/25; G01N 21/65
USPC 73/865.8; 356/51; 250/339.01; 356/301; 250/459.1

(57) ABSTRACT

A method of determining cleanliness of a manufacturing area is disclosed. The method of determining cleanliness of a manufacturing area comprising swabbing at least one target area within the manufacturing area, placing the swab in a testing apparatus, analyzing for the presence of the at least one substance with the testing apparatus and determining in real time the cleanliness of the manufacturing area.

### FIELD OF THE INVENTION

The invention generally relates to methods of determining the cleanliness of a manufacturing area.

### BACKGROUND OF THE INVENTION

The cleanliness of manufacturing areas and equipment should be ensured to minimize or eliminate any possibilities of carryover that might affect subsequent materials processed with the same equipment. Thus, the cleanliness of manufacturing areas and equipment is typically defined by a cleaning verification protocol where the methodology and limits used to determine the cleanliness are established. This need for cleaning verification is true in a number of industries including the pharmaceutical, food, petrochemical and consumer product industries. There currently are a number of regulatory guidelines related to cleaning verification, generally related to pharmaceuticals. These include guidance from e.g., FDA, Validation of cleaning processes, July 1993 and Guidance for industry: Manufacture Q7A, Good Manufacturing Practice Guidance for Active Pharmaceutical Ingredients, FDA, August 20; Health Canada, Cleaning Validation Guidelines (GUIDE-0028); PHARMA, Cleaning Validation in Active pharmaceutical Ingredient manufacturing plants, September 1999; and, ICH, ICH Good Manufacturing Practice Guideline for Active Pharmaceutical.

Cleaning verification generally refers to the process in which the cleanliness of manufacturing area and/or equipment is confirmed. Cleaning verification is typically conducted by swabbing a predefined area of the manufacturing equipment, performing a solvent extraction of the swab and analyzing the extract to determine the level of residual material. Swabs for cleaning verification are selected by the type of sample and surface to be evaluated to determine if interferences are present. Each of these is typically examined during method development and they are included in the cleaning verification process. Swabs and surfaces are prepared per the method (“samples”) and without the step of swabbing (“blanks”). Hence, both sample values and blank values are generated in the method. The number of swabs often depends on the scale of the equipment, complexity of the equipment (the more complex shapes involved will correspond to more swabbing locations due to the difficulty in cleaning these locations) and the material in process and can range from a hand full into the hundreds. Generally, high performance liquid chromatography (HPLC) is used for determining the level of residual material but other methods such as UV/visible spectroscopy, fluorescence spectroscopy and total organic carbon are also used. The output of the method for determining the cleanliness of the swab will be compared to a reference value for determining the absolute cleanliness of the manufacturing area and/or equipment. All of the aforementioned techniques have the drawback of regarding the length of time involved in analyzing the samples.

One drawback of the current cleaning verification process is the compounded time it takes from the initial swabbing to the final reporting of the level of cleanliness. Typically, the process can take a minimum of three days to turnaround. The cost of the cleaning verification process comes from a number of sources including e.g., the time for swabbing, extraction and analysis; reagents used in extraction and analysis; loss of manufacturing capacity as a result of equipment waiting for the results of the testing and the cost of recleaning if the test come back showing the equipment required further cleaning.

### SUMMARY OF THE INVENTION

In a first aspect, the invention provides a method for determining cleanliness of a manufacturing area comprising swabbing at least one target area within the manufacturing area, placing the swab in a testing apparatus, analyzing for the presence of at least one substance with the testing apparatus, and determining in real time the cleanliness of the manufacturing area.

In another aspect, the invention provides a method of determining cleanliness of a manufacturing area comprising providing a luminescence apparatus for determining the presence of at least one substance, contacting at least one target area within the manufacturing area with a swab, placing the swab in the luminescence apparatus, analyzing for the presence of the at least one substance with the luminescence apparatus, comparing the analysis to a reference value; and determining in real time the cleanliness of the manufacturing area. These and other aspects are encompassed by the present invention.

### BRIEF DESCRIPTION OF THE DRAWINGS

FIG. 1 depicts the general steps for a method for determining the cleanliness of a substance.

FIGS. 2a-2c depicts one embodiment of a testing apparatus for determining the cleanliness of a substance.

FIG. 3 depicts another embodiment of a testing apparatus for determining the cleanliness of a substance.

FIG. 4 depicts one embodiment of a method for determining the cleanliness of a substance for a plurality of samples.

FIG. 5 depicts another embodiment of a method for determining the cleanliness of a substance for a plurality of samples.

FIGS. 6a and 6b depict embodiments of a method for preparing a swab for determining the cleanliness of a substance.

FIG. 7 depicts another embodiment of a portable testing apparatus for determining the cleanliness of a substance.

FIG. 8 is an exemplary of the data produced by a testing apparatus for determining the cleanliness of a substance.

FIG. 9 is an exemplary of the data produced by a testing apparatus for determining the cleanliness of a substance.

### DETAILED DESCRIPTION OF THE INVENTION

The invention will now be described with respect to the embodiments presented herein. Before describing the present invention in detail, it is to be understood that this invention is not limited to particularly exemplified structures, apparatus, systems, materials or methods as such may, of course, vary. Thus, although a number of apparatus, systems and methods similar or equivalent to those described herein can be used in the practice of the present invention, the preferred apparatus, systems and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a”, “an”, “the” and “one” include plural referents unless the content clearly dictates otherwise. Thus, for example, reference to “a salt” includes two or more such salts; reference to “a constituent” includes two or more such constituents and the like.

Repeat use of reference characters herein and in the figures is intended to represent same or analogous features or elements of the invention.

The following terms are used herein:

“Cleanliness” is defined as both no more than 0.1% of the normal therapeutic dose of any product, at the maximum daily dose of the product, and no more than 10 ppm of any product appearing in another product and as no visible quantity of residue on the equipment after cleaning procedures are performed.

A “visible quantity” in cleanliness is generally understood to be below 1 μg/cm².

A “Swab” is generally a portion of knit material (for example polyolefin, natural fibers, polyester, quartz wool, polypropylene-cellulose-polypropylene composites and/or blends of these) that will not leave behind fibers after swabbing and contains minimal extractable materials.

“Swabbing” refers to an physical method of testing having an operator conducting a swabbing procedure where the operator follows a series of steps which generally include pretreating the swab(s) in a sample solvent, squeezing the swab(s), swabbing the surface of the tested surface firmly and evenly with one side of the swab(s) in a horizontal direction, and with the other side in a vertical direction back and forth (one stroke back and one stroke forward) to cover the entire area.

The term “real-time”, as used herein, means and includes substantially simultaneously processing data as the data is received.

Implementation of the methods and systems of embodiments of the present invention, as described herein, can involve performing or completing selected tasks or steps manually, automatically, or a combination thereof. In some embodiments of the present invention, several selected steps could be implemented by hardware or by software on any operating system or any firmware or a combination thereof. For example, as hardware, selected steps of embodiments of the invention could be implemented as a chip or a circuit. As software, selected steps of embodiments of the invention could be implemented as a plurality of software instructions being executed by a computer using any suitable operating system. In any case, selected steps of the method and system of the invention could be described as being performed by a data processor, such as a computing platform for executing a plurality of instructions.

In one aspect, the method for determining the cleanliness of a manufacturing area would use a testing apparatus for determining the presence of at least one substance. This substance generally would be selected from the group comprising UV/vis spectroscopy, near infrared spectroscopy, infrared spectroscopy, Raman spectroscopy, luminescence spectroscopy and combinations of these. In a further aspect, the testing apparatus for determining the presence of at least one substance would directly determine the presence of said at least one substance. In a further aspect, the testing apparatus for determining the presence of at least one substance would use an integrating sphere. In a further aspect, the testing apparatus for determining the presence of at least one substance would be portable. In yet a further aspect, the testing apparatus for determining the presence of at least one substance would perform an extraction to determining the presence of at least one substance.

In another aspect, the invention is further adapted to upon detection of at least one substance provide an electrical output. In a further aspect, the electrical output is a human sensible output, the human sensible output consisting of an acoustic, visual, mechanical, electrical indicator and combinations of these.

In another aspect, the invention provides a means for determining the cleanliness of a substance, where the substance is selected from the group consisting of pharmaceutical compounds, petrochemical compounds, cleaning agents, excipients, and combinations of these.

This method of cleaning a substance is generally suited to substances such as those selected from the group comprising pharmaceutical active ingredients, pharmaceutical excipients, cleaning agents, oils, surfactants and combinations of these. This method of cleaning a manufacturing area is generally suited to a manufacturing area such as those selected from the group consisting of pharmaceutical manufacturing, chemical manufacturing, petrochemical manufacturing and combinations of these. This method is especially suited to pharmaceutical manufacturing where the manufacturing is selected from the group consisting of active ingredient manufacturing, formulation manufacturing, packaging manufacturing and combinations of these.

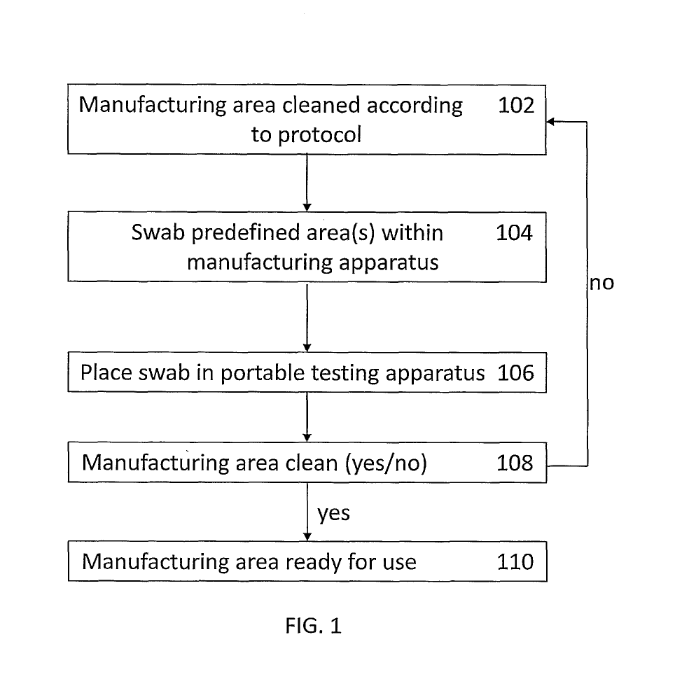

As schematically illustrated in FIG. 1, a manufacturing area is cleaned according to protocol (102) and the cleanliness is determined by using a testing apparatus to determine the presence of at least one substance. The method would include swabbing at least one target area within the manufacturing area (104), placing the swab(s) in the testing apparatus (106), and determining if the area is clean (108) with the testing apparatus. If the area is determined to be clean, the area would be ready for use (110) and if not would require further cleaning and swabbing (104). Methods for determining if the area was clean from the signal generated would include direct analysis of signal generated, comparison of the signal generated to a reference signal, performing a mathematical function on the generated signal and combinations of these. Methods for determining if the area was clean from the signal generated are described in texts such as Analytical Chemistry, by Gary Christian, John Wiley and Sons and Chemical Instrumentation: A Systematic Approach, by Howard Strobel and William Heineman, John Wiley and Sons. Methods for the comparison of the generated signal would include applying look up table, an electrical bridge, a signal threshold value and combinations of these. Methods for performing a mathematical function would include addition, subtraction, division, multiplication, integration, derivatives, curve fitting, statistical analysis and combinations of these.

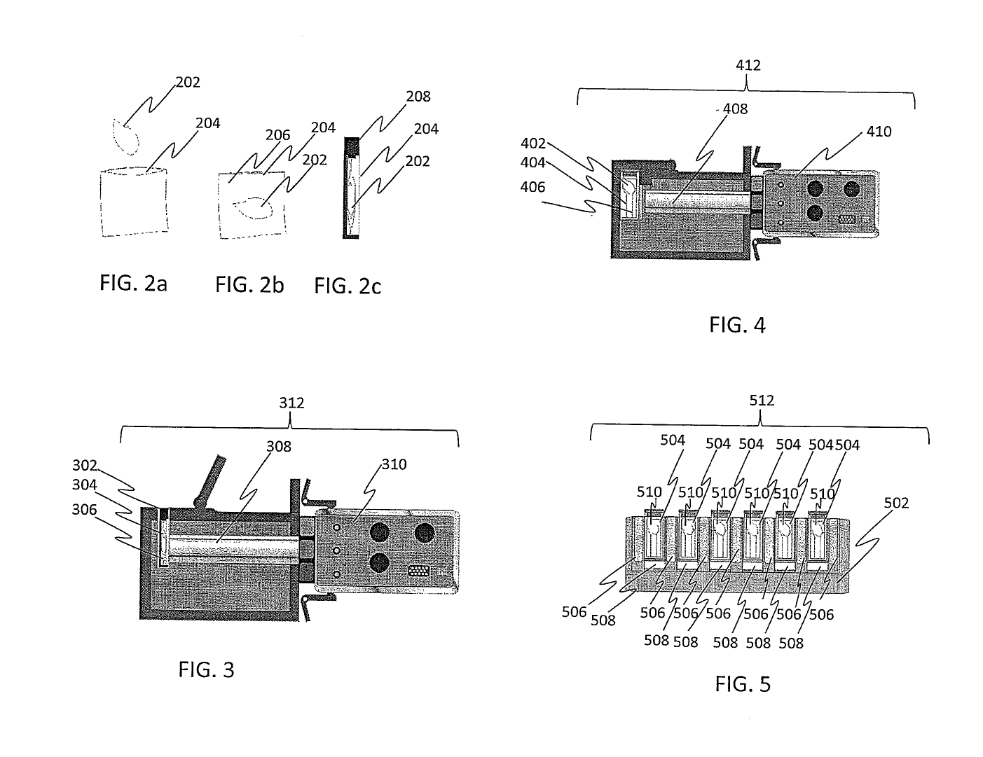

FIGS. 2a-2c are illustrative of one method for determining the cleanliness of a manufacturing area where a swab (202) is used to swab an area of the manufacturing area. Post swabbing, the swab (202) would be stored in a secondary container (204), as shown in FIG. 2a. Examples of a secondary container would include a plastic bag, a bottle, a vial and a cuvette. The swab (202) would be sealed (206) in the secondary container (204), as depicted in FIG. 2b. The secondary container (204) could be subsequently placed in a sample holder (208) for analysis of the swab (202) as shown in FIG. 2c.

FIG. 3 is illustrative of a testing apparatus (312) for determining the presence of at least one substance directly. In this example, the swab (302) is in a secondary container (304) with both being held in a sample holder (306). The sample holder (306) is held in the testing apparatus (312) and a sample interface (308) resides between the sample holder (306) and analytical unit (310). Embodiment of the testing apparatus could include the sample holder (306), analytical unit (310), computer, cart, vials, swabs, solvent storage and waste storage. Examples of the analytical unit (310) would include UV/vis spectroscopy, near infrared spectroscopy, infrared spectroscopy, Raman spectroscopy, luminescence spectroscopy and combinations thereof. Optionally, an integrating sphere could be used with the analytical unit (310).

FIG. 4 is illustrative of a testing apparatus with an extraction to (412) for determining the presence of at least one substance. In this example, the swab (402) is in a secondary container (404) capable of holding both the swab (402) and an extraction medium with both being held in a sample holder (406). Examples of an extraction medium would include an organic solvent, water, surfactants and supercritical fluids. It is to be understood that the extraction could involve manual intervention, be automated and combinations of these. The sample holder (406) is held in the portable testing apparatus (412) and a sample interface (408) resides between the sample holder (406) and analytical unit (410). Examples of the analytical unit (410) would include UV/vis spectroscopy, near infrared spectroscopy, infrared spectroscopy, Raman spectroscopy, luminescence spectroscopy and combinations of these. Optionally, an integrating sphere could be used with the analytical unit (410).

FIG. 5 is illustrative of a method for determining the presence of at least one substance with a testing device (512) for at least one swab (510). In this example, at least one swab (510) is placed into a secondary container (504) which could contain an extraction medium which is held in a sample holder (502). To facilitate the extraction optional heating and/or cooling could be used (506), for example by a liquid jacket, peltier element, resistance element and combinations of these along with optional agitation (508) by a stir bar, ultrasonic source, shaker and combinations of these.

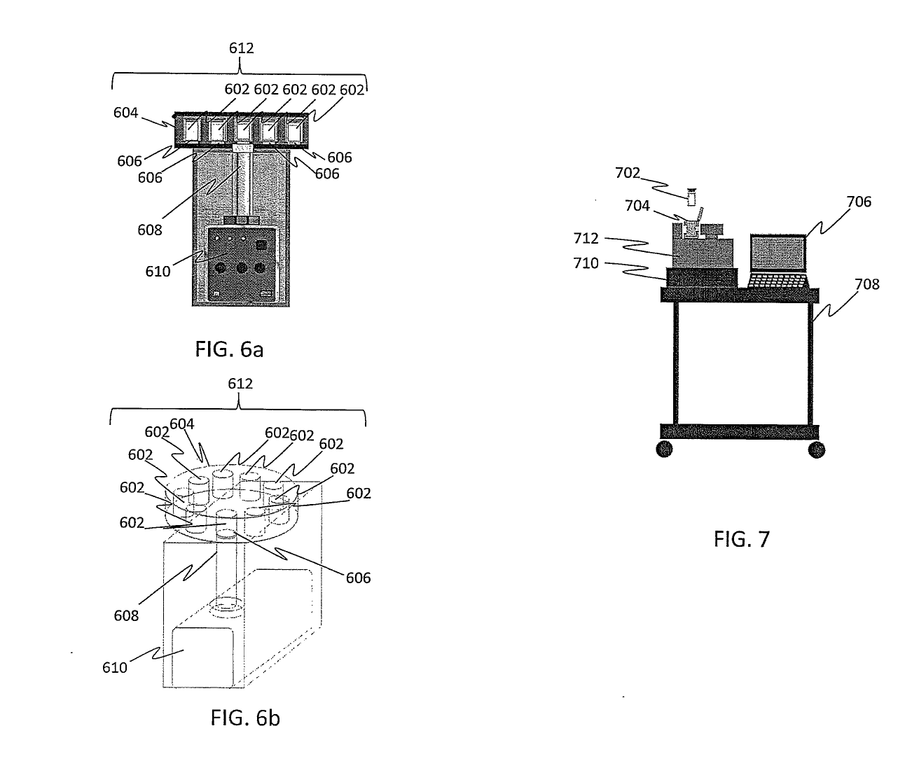

FIG. 6a is illustrative of a method for determining the presence of at least one substance using a testing apparatus (612) from at least one secondary container (602). In this example, at least one secondary container (602) is held is a sample holder (604). The sample rack may contain an interface window (606) made of a suitable material such as glass, quartz, sapphire, diamond and combinations of these. The sample holder (604) is held in the testing apparatus (612) and a sample interface (608) resides between the sample holder (604) and analytical unit (610). Examples of the analytical unit (610) would include UV/vis spectroscopy, near infrared spectroscopy, infrared spectroscopy, Raman spectroscopy, luminescence spectroscopy and combinations of these.

FIG. 6b is illustrative of a method for determining the presence of at least one substance using a testing apparatus (612) from at least one secondary container (610). In this example, at least one secondary container (602) is held is a sample holder (604). As depicted in FIG. 6b the sample holder (604) could be circular and also could be a track, a straight line and combinations of these. The sample rack may contain an interface window (606) made of a suitable material such as glass, quartz, sapphire, diamond and combinations of these. The sample holder (604) is held in the testing apparatus (612) and a sample interface (608) resides between the sample holder (604) and analytical unit (610). Examples of the analytical unit (610) would include UV/vis spectroscopy, near infrared spectroscopy, infrared spectroscopy, Raman spectroscopy, luminescence spectroscopy and combinations of these.

FIG. 7 is illustrative of a method for determining the presence of at least one substance using a portable testing apparatus (712). In this example, at least one secondary container (702) is held is a sample holder (704). The portable testing apparatus (712) is shown in combination with a computer (706) which is carried upon a cart (708) along with a storage unit holding sample preparation equipment (710). In this example the cart (708) could be rolled out to the manufacturing area and the portable testing apparatus (712) used to determine the cleanliness of manufacturing equipment.

### Example 1

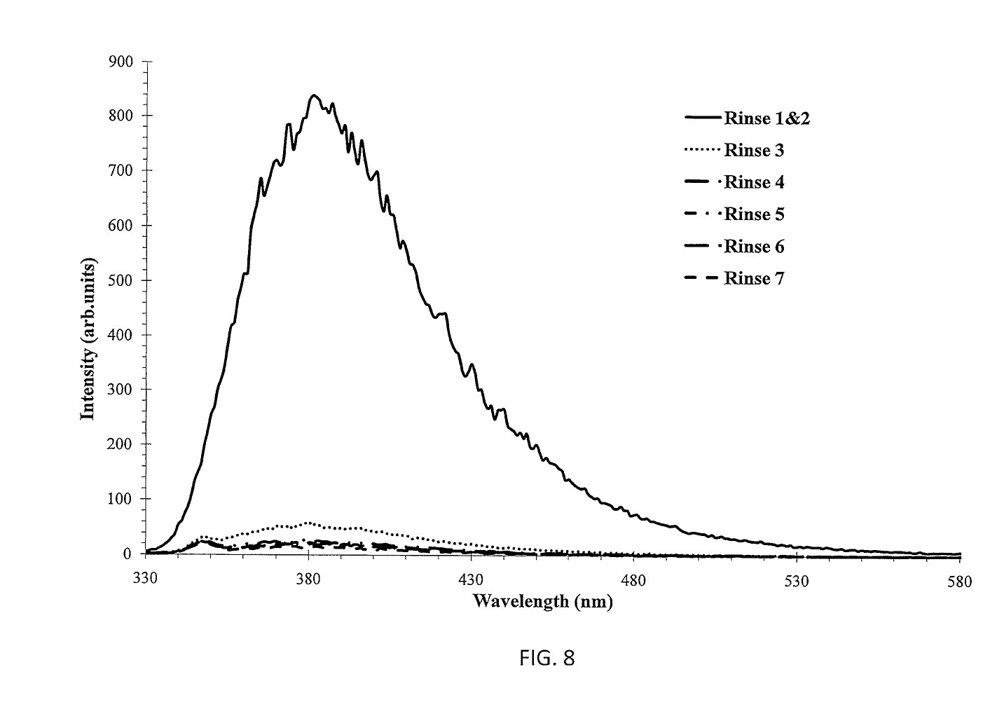

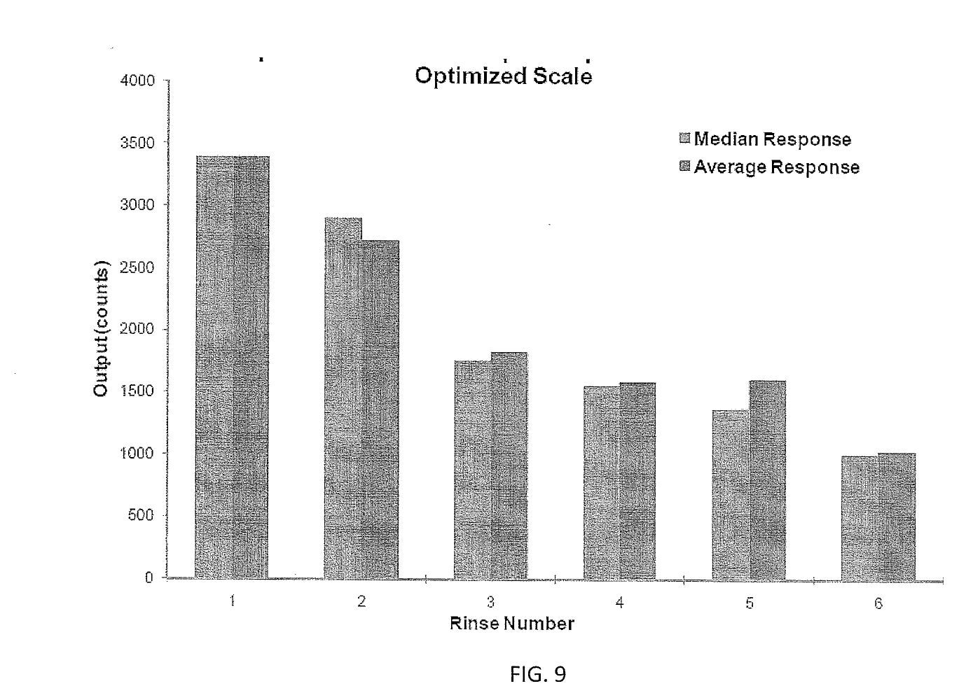

A stainless steel coupon was contaminated with Rosiglitazone and rinsed with sequential 100 mL aliquots of distilled water. Both the collected rinse and stainless steel coupon were then subjected to luminescence analysis. The rinse was analyzed with a Cary Eclipse Fluorometer (Agilent Technology, Santa Clara, Calif.) and the coupon analyzed by the method of the invention and apparatus, illustrated in FIG. 3. The intensity of the luminescence signal for Rosiglitazone using both analytical methods as a function of rinse number was collected, as shown in FIGS. 8 and 9.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

What is claimed is:

1. A method of determining cleanliness of a manufacturing area, comprising:
swabbing at least one target area within said manufacturing area;
placing said swab in a testing apparatus;
analyzing for the presence of said at least one substance with said testing apparatus; and
determining in real time the cleanliness of said manufacturing area.

2. The method of claim 1, wherein said at least one substance is selected from the group comprising pharmaceutical active ingredients, pharmaceutical excipients, cleaning agents, oils, surfactants and combinations thereof.

3. The method of claim 1, wherein said manufacturing area is selected from the group consisting of pharmaceutical manufacturing, chemical manufacturing, petrochemical manufacturing and combinations thereof.

4. The method of claim 3, wherein said pharmaceutical manufacturing is selected from the group consisting of active ingredient manufacturing, formulation manufacturing, packaging manufacturing and combinations thereof.

5. The method of claim 1, wherein said testing apparatus for determining the presence of at least one substance is selected from the group comprising UV/vis spectroscopy, near infrared spectroscopy, infrared spectroscopy, Raman spectroscopy, luminescence spectroscopy and combinations thereof.

6. The method of claim 1, wherein said testing apparatus for determining the presence of at least one substance is portable.

7. The method of claim 1, wherein said testing apparatus for determining the presence of at least one substance is luminescence spectroscopy.

8. The method of claim 1, wherein said testing apparatus for analyzing the presence of at least one substance is configured to directly analyze for the presence of said at least one substance.

9. The method of claim 1, wherein said testing apparatus for analyzing the presence of at least one substance is configured to perform an extraction to analyze for the presence of said at least one substance.

10. The method of claim 8, wherein said testing apparatus for analyzing the presence of at least one substance is further adapted to upon detection of said at least one substance provide an electrical output.

11. The method of claim 10, wherein said electrical output is a human sensible output, said human sensible output consisting of an acoustic, visual, mechanical, electrical indicator and combinations thereof.

12. The method of claim 8, wherein said at least one substance is selected from the group consisting of pharmaceutical compounds, petrochemical compounds, cleaning agents, excipients, and combinations thereof.

13. A method of determining cleanliness of a manufacturing area, comprising:
providing a luminescence apparatus for determining the presence of at least one substance;
contacting at least one target area within said manufacturing area with a swab
placing said swab in said luminescence apparatus;
analyzing for the presence of said at least one substance with said luminescence apparatus;
comparing said analysis to a reference value;
calculating the presence of at least one substance from the manufacturing area by using the area represented by the swabbed target area and
determining in real time the cleanliness of said manufacturing area.

14. The method of claim 13, wherein said at least one substance is selected from the group comprising pharmaceutical active ingredients, pharmaceutical excipients, cleaning agents, oils, surfactants and combinations thereof.

15. The method of claim 13, wherein said manufacturing area is selected from the group consisting of pharmaceutical manufacturing, chemical manufacturing, petrochemical manufacturing and combinations thereof.

16. The method of claim 13, wherein said pharmaceutical manufacturing is selected from the group consisting of active ingredient manufacturing, formulation manufacturing, packaging manufacturing and combinations thereof.

17. The method of claim 13, wherein said testing apparatus for analyzing the presence of at least one substance directly analyzes for the presence of said at least one substance.

18. The method of claim 13, wherein said testing apparatus for analyzing the presence of at least one substance performs an extraction to analyze for the presence of said at least one substance.

19. The method of claim 17, wherein said testing apparatus for analyzing the presence of at least one substance is further adapted to upon detection of said at least one substance provides an electrical output.

20. The method of claim 19, wherein said electrical output is a human sensible output, said human sensible output consisting of an acoustic, visual, mechanical, electrical indicator and combinations thereof.

21. The method of claim 17, wherein said at least one substance is selected from the group consisting of pharmaceutical compounds, petrochemical compounds, cleaning agents, excipients, and combinations thereof.

22. The method of claim 13, wherein said testing apparatus for analyzing the presence of at least one substance is portable.
